(19) United States
(12) Patent Application Publication
(10) Pub. No.: US 2012/0189620 A1
(43) Pub. Date: Jul. 26, 2012

(54) METHODS FOR TREATING NON-FUNCTIONING PITUITARY ADENOMA

(75) Inventor: Nelson M. Oyesiku, Atlanta, GA (US)

(73) Assignee: EMORY UNIVERSITY, Atlanta, GA (US)

(21) Appl. No.: 13/354,456

(22) Filed: Jan. 20, 2012

Related U.S. Application Data
(60) Provisional application No. 61/435,009, filed on Jan. 21, 2011.

(51) Int. Cl. A61K 39/395; A61P 35/00

(52) U.S. Cl. 424/133.1; 424/174.1

(57) ABSTRACT

The disclosure is directed to methods for treating non-functioning pituitary adenomas with alpha-folate receptor (“FR-α”) and compositions. In some embodiments, the disclosure is directed to a method for treating a pituitary adenoma that includes administering farletuzumab to a subject diagnosed with a non-functioning pituitary adenoma.

### CROSS REFERENCE TO RELATED APPLICATIONS

This non-provisional application claims priority to U.S. provisional application No. 61/435,009 filed Jan. 21, 2011, hereby incorporated by reference in its entirety.

### FIELD

This disclosure relates to using purified monoclonal antibodies that specifically bind to the alpha-folate receptor (“FR-α”) and compositions thereof to treat certain non-malignant tumors. In some embodiments, the disclosure also relates to methods of treating non-functioning pituitary adenomas, preoperatively or postoperatively, using antibodies that block the biological activity of FR-α.

### BACKGROUND

Pitutary adenomas are frequently occurring intracranial tumors. Functional pituitary adenomas produce specific hormones and can cause life-threatening diseases, infertility, and impotence. Non-functioning pituitary adenomas, the most common type of pituitary tumors, do not secrete hormones. These adenomas can cause progressive visual loss, headaches and symptoms of pituitary dysfunction through compression of regional structures. However, unlike functional pituitary adenomas, there is no available effective medical therapy for non-functioning pituitary adenomas.

Currently, transsphenoidal surgical resection of the adenoma has been choice therapy for adenomas>1 cm in size because many will grow in size over a few years without treatment. However, gross total resection is not available in about 50% of the cases due to the tumor invasion of surrounding anatomical structures or large tumor size. Surgery can also be challenging because of the adenomas proximity to the carotid artery and optic nerve, and their invasiveness. Also, of those cases that receive surgery, up to 50% will suffer disease recurrence. Thus, there is a need for a non-surgical and less invasive medical therapy for slowing or stopping growth of non-functioning pituitary adenomas.

FR-α gene was shown to be significantly overexpressed in non-functioning adenomas compared to control normal pituitary or prolactin- and growth hormone-secreting pituitary adenomas. Evans et al. (2001) J Clin Endocrinol Metab 86:3097-107. In vivo imaging with ^{99m}Tc-EC20, a folate receptor targeted radiotracer, revealed moderate to marked radiotracer uptake in a significant proportion of pituitary tumors further indicating a biological role for this receptor in pituitary tumors. Fisher et al. (2008) J Nucl Med 19:899-906 and Galt et al. (2010) J Nucl Med 51:1716-1723.

Antibodies that specifically bind to the FR-α have been shown to have antitumor activity in epithelial ovarian cancer. Specifically, farletuzumab, a humanized monoclonal antibody against FR-α is being used in clinical trials to treat epithelial ovarian cancer. See Konner et al., Clin Cancer Res 2010 16(21): 5288-95 and U.S. Patent Application Publication No. 2009/0274697.

### SUMMARY

The disclosure relates to the methods for treating non-functioning pituitary adenoma. In some embodiments, the disclosure relates to methods of administering antibodies that specifically bind to alpha-folate receptor to treat non-functioning pituitary adenoma.

In some embodiments, the disclosure relates to administering farletuzumab to treat a subject diagnosed with a non-functioning pituitary adenoma.

In some embodiments, the subject underwent a medical procedure resecting a portion of the pituitary adenoma. In certain embodiments, farletuzumab is administered before the medical procedure. In other embodiments, farletuzumab is administered after said medical procedure. In further embodiments, farletuzumab is administered before and after said medical procedure

In some embodiments, farletuzumab is administered by intravenous infusion. In certain embodiments, farletuzumab is administered between 10.0 mg/kg and 1.0 mg/kg. In other embodiments, farletuzumab is administered between 5.0 mg/kg and 2.5 mg/kg.

In some embodiments, farletuzumab is administered in combination with a second anti-tumor agent. In further embodiments, farletuzumab is administered to induce a therapeutic effect or in an effective amount to inhibit the growth of the non-functioning pituitary adenoma.

### TERMS

The term “pituitary adenoma” refers to a benign tumor that occurs in pituitary gland.

The term “non-functioning tumors” or “non-functioning pituitary adenoma” refers to a tumor that does not secrete any active hormone.

The term “preventing” refers to decreasing the probability that an organism contracts or develops an abnormal condition. An abnormal condition includes a benign tumor, such as a pituitary adenoma.

The term “treating” refers to having a therapeutic effect and at least partially alleviating or abrogating an abnormal condition in the organism. Treating includes reduction in tumor growth, inhibition of tumor growth and maintenance of inhibited tumor growth.

The term “therapeutic effect” refers to the inhibition of an abnormal condition. A therapeutic effect relieves to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) inhibition (i.e., slowing or stopping) of growth of tumor cells in vivo (c) promotion of cell death; (d) inhibition of degeneration; (e) relieving to some extent one or more of the symptoms associated with the abnormal condition; and (f) enhancing the function of a population of cells.

As used herein “blocks a biological activity of FR-α” refers to the ability of antibodies (or fragments thereof) to prevent folate binding to FR-α, to prevent the uptake of folate by cells, or to inhibit signal transduction in the cell triggered by folate.

As used herein, the term “about” refers to an approximation of a stated value within an acceptable range. Preferably the range is +/−5% of the stated value.

As used herein, the term “FR-α binding competitors” refers to aberrant transcripts of the nucleic acids encoding antibodies and aberrant translation products that do not have the biological properties of anti-FR-α antibodies (e.g., antigen binding affinity, ability to block a biological activity of FR-α). For example, an aberrant transcript may contain a deletion, a frameshift, a nonsense mutation, or a missense mutation. An example of an aberrant translation product is an alternative splice variant.

### Antibodies

As used herein, the term “antibodies” refers to antibodies that specifically bind FR-α. The antibodies may specifically bind a monomeric form of FR-α. The antibodies may specifically bind a multimeric form of FR-α (e.g., a tetrameric form) and not the monomeric form of FR-α. Antibodies may also block a biological activity of FR-α. The antibodies may also block a biological activity of FR-α on FR-α-bearing cells. Antibodies preferably induce antibody-dependent cellular cytotoxicity (ADCC) of FR-α-bearing cells.

Preferred antibodies, and antibodies suitable for use in the methods of the disclosure, include, for example, fully human antibodies, human antibody homologs, humanized antibody homologs, chimeric antibody homologs, Fab, Fab′, F(ab′)₂ and F(v) antibody fragments, single chain antibodies, and monomers or dimers of antibody heavy or light chains or mixtures thereof. Antibodies may include monoclonal antibodies. Examples of such antibodies include those discussed in U.S. patent application Ser. No. 12/500,144 filed on Jul. 9, 2009, which is a divisional of U.S. patent application Ser. No. 11/056,776 filed on Feb. 11, 2005. These patent applications are incorporated in their entirety. Also, an example of such antibodies includes farletuzumab, also called MORAb-003. Farletuzumab is a humanized IgG1 antibody that targets FR-α.

The antibodies may include intact immunoglobulins of any isotype including types IgA, IgG, IgE, IgD, IgM (as well as subtypes thereof). The antibodies may include intact IgG and more preferably IgG1. The light chains of the immunoglobulin may be kappa or lambda.

The antibodies may include portions of intact antibodies that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. Thus, antigen binding fragments, as well as full-length dimeric or trimeric polypeptides derived from the above-described antibodies are themselves useful.

A “chimeric antibody” is an antibody produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody. One approach, described in EP 0239400 to Winter et al. describes the substitution of one species' complementarity determining regions (CDRs) for those of another species, such as substituting the CDRs from human heavy and light chain immunoglobulin variable region domains with CDRs from mouse variable region domains. These altered antibodies may subsequently be combined with human immunoglobulin constant regions to form antibodies that are human except for the substituted murine CDRs which are specific for the antigen. Methods for grafting CDR regions of antibodies may be found, for example in Riechmann et al. (1988) Nature 332:323-327 and Verhoeyen et al. (1988) Science 239:1534-1536.

The direct use of rodent monoclonal antibodies (MAbs) as human therapeutic agents led to human anti-rodent antibody (“HARA”) (for example, human anti-mouse antibody (“HAMA”)) responses which occurred in a significant number of patients treated with the rodent-derived antibody (Khazaeli, et al., (1994) Immunother. 15:42-52). Chimeric antibodies containing fewer murine amino acid sequences are believed to circumvent the problem of eliciting an immune response in humans.

Refinement of antibodies to avoid the problem of HARA responses led to the development of “humanized antibodies.” Humanized antibodies are produced by recombinant DNA technology, in which at least one of the amino acids of a human immunoglobulin light or heavy chain that is not required for antigen binding has been substituted for the corresponding amino acid from a nonhuman mammalian immunoglobulin light or heavy chain. For example, if the immunoglobulin is a mouse monoclonal antibody, at least one amino acid that is not required for antigen binding is substituted using the amino acid that is present on a corresponding human antibody in that position. Without wishing to be bound by any particular theory of operation, it is believed that the “humanization” of the monoclonal antibody inhibits human immunological reactivity against the foreign immunoglobulin molecule.

As a non-limiting example, a method of performing complementarity determining region (CDR) grafting may be performed by sequencing the mouse heavy and light chains of the antibody of interest that binds to the target antigen (e.g., FR-α) and genetically engineering the CDR DNA sequences and imposing these amino acid sequences to corresponding human V regions by site directed mutagenesis. Human constant region gene segments of the desired isotype are added, and the “humanized” heavy and light chain genes are co-expressed in mammalian cells to produce soluble humanized antibody. A typical expression cell is a Chinese Hamster Ovary (CHO) cell. Suitable methods for creating the chimeric antibodies may be found, for example, in Jones et al. (1986) Nature 321:522-525; Riechmann (1988) Nature 332:323-327; Queen et al. (1989) Proc. Nat. Acad. Sci. USA 86:10029; and Orlandi et al. (1989) Proc. Natl. Acad. Sci. USA 86:3833.

Queen et al. (1989) Proc. Nat. Acad. Sci. USA 86:10029-10033 and WO 90/07861 describe the preparation of a humanized antibody. Human and mouse variable framework regions were chosen for optimal protein sequence homology. The tertiary structure of the murine variable region was computer-modeled and superimposed on the homologous human framework to show optimal interaction of amino acid residues with the mouse CDRs. This led to the development of antibodies with improved binding affinity for antigen (which is typically decreased upon making CDR-grafted chimeric antibodies). Alternative approaches to making humanized antibodies are known in the art and are described, for example, in Tempest (1991) Biotechnology 9:266-271.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The antibodies may be used alone or as immunoconjugates with a cytotoxic agent. In some embodiments, the agent is a chemotherapeutic agent. In some embodiments, the agent is a radioisotope, including, but not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, and fissionable nuclides such as Boron-10 or an Actinide. In other embodiments, the agent is a toxin or cytotoxic drug, including but not limited to ricin, modified Pseudomonas enterotoxin A, calicheamicin, adriamycin, 5-fluorouracil, and the like. Methods of conjugation of antibodies and antibody fragments to such agents are known in the literature.

The antibodies may include derivatives that are modified, e.g., by the covalent attachment of any type of molecule to the antibody such that covalent attachment does not prevent the antibody from binding to its epitope. Examples of suitable derivatives include, but are not limited to fucosylated antibodies and fragments, glycosylated antibodies and fragments, acetylated antibodies and fragments, pegylated antibodies and fragments, phosphorylated antibodies and fragments, and amidated antibodies and fragments. The antibodies and derivatives thereof may themselves by derivatized by known protecting/blocking groups, proteolytic cleavage, linkage to a cellular ligand or other proteins, and the like. At least one heavy chain of the antibody may be fucosylated, such as the N-linked. At least one heavy chain of the antibody may comprise a fucosylated, N-linked oligosaccharide.

The antibodies may include variants having single or multiple amino acid substitutions, deletions, additions, or replacements that retain the biological properties (e.g., block a biological activity of FR-α, binding affinity) of the antibodies. The skilled person can produce variants having single or multiple amino acid substitutions, deletions, additions or replacements. These variants may include, inter alia: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies may include variants in which amino acid residues from one species are substituted for the corresponding residue in another species, either at the conserved or nonconserved positions. In another embodiment, amino acid residues at nonconserved positions are substituted with conservative or nonconservative residues. The techniques for obtaining these variants, including genetic (suppressions, deletions, mutations, etc.), chemical, and enzymatic techniques, are known to the person having ordinary skill in the art. Antibodies may also include antibody fragments. A “fragment” refers to polypeptide sequences which are preferably at least about 40, more preferably at least to about 50, more preferably at least about 60, more preferably at least about 70, more preferably at least about 80, more preferably at least about 90, and more preferably at least about 100 amino acids in length, and which retain some biological activity or immunological activity of the full-length sequence, for example, the ability to block a biological activity of FR-α and/or FR-α binding affinity.

The antibodies may include fully human antibodies such as those derived from peripheral blood mononuclear cells of ovarian, breast, renal, colorectal, lung, endometrial, or brain cancer patients. Such cells may be fused with myeloma cells, for example, to form hybridoma cells producing fully human antibodies against FR-α.

The antibody may comprise a light chain
comprising an amino acid sequence of
SEQ ID NO: 1:
DIQLTQSPSSLSASVGDRVTITCSVSSSISSNNLHWYQQKPGKAPKPWIY
GTSNPASGVPSRFSGSGSGTDYTFTISSLQPEDIATYYCQQWSSYPYMYT
FGQGTKVEIK.
The antibody may also comprise a light chain
comprising an amino acid sequence of
SEQ ID NO: 2:
DIQLTQSPSSLSASVGDRVTITCSVSSSISSNNLHWYQQKPGKAPKPWIY
GTSNPASGVPSRFSGSGSGTDYTFTISSLQPEDIATYYCQQWSSYPYMYT
FGQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC.
The antibody may also comprise a light chain
comprising an amino acid sequence of
SEQ ID NO: 3:
MGWSCIILFLVATATGVHSDIQLTQSPSSLSASVGDRVTITCSVSSSISS
NNLHWYQQKPGKAPKPWIYGTSNPASGVPSRFSGSGSGTDYTFTISSLQP
EDIATYYCQQWSSYPYMYTFGQGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC
(leader sequence underlined).
Also antibodies may comprise a heavy chain
comprising an amino acid sequence of
SEQ ID NO: 4:
EVQLVESGGGVVQPGRSLRLSCSASGFTFSGYGLSWVRQAPGKGLEWVAM
ISSGGSYTYYADSVKGRFAISRDNAKNTLFLQMDSLRPEDTGVYFCARHG
DDPAWFAYWGQGTPVTVSS.
The antibodies may also comprise a heavy chain
comprising an amino acid sequence of
SEQ ID NO: 5:
EVQLVESGGGVVQPGRSLRLSCSASGFTFSGYGLSWVRQAPGKGLEW
VAM ISSGGSYTYYADSVKGRFAISRDNAKNTLFLQMDSLRPEDTGVYFC
ARHGDDPAWFAYWGQGTPVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK.
The heavy chain of the antibody may comprise
an amino acid sequence of
SEQ ID NO: 6:
MGWSCIILFLVATATGVHSEVQLVESGGGVVQPGRSLRLSCSASGFTFSG
YGLSWVRQAPGKGLEWVAMISSGGSYTYYADSVKGRFAISRDNAKNTLFL
QMDSLRPEDTGVYFCARHGDDPAWFAYWGQGTPVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGK
(leader sequence underlined).

The antibody may also comprise a heavy chain comprising an amino acid sequence of SEQ ID NO:4, 5, or 6 and a light chain comprising an amino acid sequence of SEQ ID NO:1, 2, or 3. For example, the antibody may comprise a heavy chain comprising an amino acid sequence of SEQ ID NO:5 and a light chain comprising an amino acid sequence of SEQ ID NO:2. The antibody may also comprise a heavy chain comprising an amino acid sequence SEQ ID NO:6 and a light chain comprising an amino acid sequence of SEQ ID NO:3.

The antibodies are preferably nontoxic as demonstrated, for example, in in vivo toxicology studies.

The antibodies and derivatives thereof may have binding affinities that include a dissociation constant (K_{d}) of less than 1×10⁻². In some embodiments, the K_{d} is less than 1×10⁻³. In other embodiments, the K_{d} is less than 1×10⁻⁴. In some embodiments, the K_{d} is less than 1×10.sup.-5. In still other embodiments, the K_{d} is less than 1×10⁻⁶. In other embodiments, the K_{d} is less than 1×10⁻⁷. In other embodiments, the K_{d} is less than 1×10.⁻⁸. In other embodiments, the K_{d} is less than 1×10⁻⁹. In other embodiments, the K_{d} is less than 1×10.sup.-10. In still other embodiments, the K_{d} is less than 1×10⁻¹¹. In some embodiments, the K_{d} is less than 1×10⁻¹². In other embodiments, the K_{d} is less than 1×10⁻¹³. In other embodiments, the K_{d} is less than 1×10⁻¹⁴. In still other embodiments, the K_{d} is less than 1×10⁻¹⁵.

Without wishing to be bound by any particular theory, the antibodies may bind the multimeric form of FR-α due to an increased avidity of the antibody as both “arms” of the antibody (Fab fragments) bind to separate FR-α molecules that make up the multimer. This leads to a decrease in the dissociation (KO of the antibody and an overall increase in the observed affinity (K_{d}).

### Methods of Producing Antibodies to FR-α

Antibodies may be produced in vivo or in vitro. One strategy for generating antibodies against FR-α involves immunizing animals with FR-α. If the animals are immunized with the monomeric or multimeric form of FR-α, the animals will produce antibodies against the protein. Standard methods are known for creating monoclonal antibodies including, but are not limited to, the hybridoma technique (see Kohler & Milstein, (1975) Nature 256:495-497); the trioma technique; the human B-cell hybridoma technique (see Kozbor et al. (1983) Immunol Today 4:72) and the EBV hybridoma technique to produce human monoclonal antibodies (see Cole, et al. in MONOCLONAL ANTIBODIES AND CANCER THERAPY, Alan R. Liss, Inc., 1985, pp. 77-96).

FR-α may be purified from cells or from recombinant systems using a variety of well-known techniques for isolating and purifying proteins. For example, but not by way of limitation, FR-α may be isolated based on the apparent molecular weight of the protein by running the protein on an SDS-PAGE gel and blotting the proteins onto a membrane. Thereafter, the appropriate size band corresponding to FR-α may be cut from the membrane and used as an immunogen in animals directly, or by first extracting or eluting the protein from the membrane. As an alternative example, the protein may be isolated by size-exclusion chromatography alone or in combination with other means of isolation and purification.

Methods for producing monoclonal antibodies that specifically bind to the multimeric form of FR-α may also be used. Multimeric, for example tetrameric, FR-α may be purified from cells or from recombinant systems using a variety of well-known techniques for isolating and purifying proteins. For example, but not by way of limitation, multimeric FR-α may be isolated based on the apparent molecular weight of the protein by running the protein on an SDS-PAGE gel and blotting the proteins onto a membrane. Thereafter, the appropriate size band corresponding to the multimeric form of FR-α may be cut from the membrane and used as an immunogen in animals directly, or by first extracting or eluting the protein from the membrane. As an alternative example, the protein may be isolated by size-exclusion chromatography alone or in combination with other means of isolation and purification.

Other means of purification are available in such standard reference texts as Zola, MONOCLONAL ANTIBODIES: PREPARATION AND USE OF MONOCLONAL ANTIBODIES AND ENGINEERED ANTIBODY DERIVATIVES (BASICS: FROM BACKGROUND TO BENCH) Springer-Verlag Ltd., New York, 2000; BASIC METHODS IN ANTIBODY PRODUCTION AND CHARACTERIZATION, Chapter 11, “Antibody Purification Methods,” Howard and Bethell, Eds., CRC Press, 2000; ANTIBODY ENGINEERING (SPRINGER LAB MANUAL), Kontermann and Dubel, Eds., Springer-Verlag, 2001.

For in vivo antibody production, animals are generally immunized with FR-α or an immunogenic portion of FR-α. The antigen is generally combined with an adjuvant to promote immunogenicity. Adjuvants vary according to the species used for immunization. Examples of adjuvants include, but are not limited to: Freund's complete adjuvant (“FCA”), Freund's incomplete adjuvant (“FIA”), mineral gels (e.g., aluminum hydroxide), surface active substances (e.g., lysolecithin, pluronic polyols, polyanions), peptides, oil emulsions, keyhole limpet hemocyanin (“KLH”), dinitrophenol (“DNP”), and potentially useful human adjuvants such as Bacille Calmette-Guerin (“BCG”) and corynebacterium parvum. Such adjuvants are also well known in the art.

Immunization may be accomplished using well-known procedures. The dose and immunization regimen will depend on the species of mammal immunized, its immune status, body weight, and/or calculated surface area, etc. Typically, blood serum is sampled from the immunized mammals and assayed for anti-FR-α antibodies using appropriate screening assays as described below, for example.

A common method for producing humanized antibodies is to graft CDR sequences from a MAb (produced by immunizing a rodent host) onto a human Ig backbone, and transfection of the chimeric genes into Chinese Hamster Ovary (CHO) cells which in turn produce a functional Ab that is secreted by the CHO cells (Shields, R. L., et al. (1995) Anti-IgE monoclonal antibodies that inhibit allergen-specific histamine release. Int Arch. Allergy Immunol. 107:412-413). The methods described within this application are also useful for generating genetic alterations within Ig genes or chimeric Igs transfected within host cells such as rodent cell lines, plants, yeast and prokaryotes (Frigerio L, et al. (2000) Assembly, secretion, and vacuolar delivery of a hybrid immunoglobulin in plants. Plant Physiol. 123:1483-1494).

Splenocytes from immunized animals may be immortalized by fusing the splenocytes (containing the antibody-producing B cells) with an immortal cell line such as a myeloma line. Typically, myeloma cell line is from the same species as the splenocyte donor. The immortal cell line may be sensitive to culture medium containing hypoxanthine, aminopterin and thymidine (“HAT medium”). The myeloma cells may be negative for Epstein-Barr virus (EBV) infection. The myeloma cells may be HAT-sensitive, EBV negative and Ig expression negative. Any suitable myeloma may be used. Murine hybridomas may be generated using mouse myeloma cell lines (e.g., the P3-NS1/1-Ag4-1, P3-x63-Ag8.653 or Sp2/O-Ag14 myeloma lines). These murine myeloma lines are available from the ATCC. These myeloma cells are fused to the donor splenocytes polyethylene glycol (“PEG”), preferably 1500 molecular weight polyethylene glycol (“PEG 1500”). Hybridoma cells resulting from the fusion are selected in HAT medium which kills unfused and unproductively fused myeloma cells. Unfused splenocytes die over a short period of time in culture. The myeloma cells may not express immunoglobulin genes.

Hybridomas producing a desired antibody that are detected by screening assays such as those described below may be used to produce antibodies in culture or in animals. For example, the hybridoma cells may be cultured in a nutrient medium under conditions and for a time sufficient to allow the hybridoma cells to secrete the monoclonal antibodies into the culture medium. These techniques and culture media are well known by those skilled in the art. Alternatively, the hybridoma cells may be injected into the peritoneum of an unimmunized animal. The cells proliferate in the peritoneal cavity and secrete the antibody, which accumulates as ascites fluid. The ascites fluid may be withdrawn from the peritoneal cavity with a syringe as a rich source of the monoclonal antibody.

Another non-limiting method for producing human antibodies is described in U.S. Pat. No. 5,789,650 desclosing transgenic mammals that produce antibodies of another species (e.g., humans) with their own endogenous immunoglobulin genes being inactivated. The genes for the heterologous antibodies are encoded by human immunoglobulin genes. The transgenes containing the unrearranged immunoglobulin encoding regions are introduced into a non-human animal. The resulting transgenic animals are capable of functionally rearranging the transgenic immunoglobulin sequences and producing a repertoire of antibodies of various isotypes encoded by human immunoglobulin genes. The B-cells from the transgenic animals are subsequently immortalized by any of a variety of methods, including fusion with an immortalizing cell line (e.g., a myeloma cell).

Antibodies against FR-α may also be prepared in vitro using a variety of techniques known in the art. For example, but not by way of limitation, fully human monoclonal antibodies against FR-α may be prepared by using in vitro-primed human splenocytes (Boerner et al. (1991) J. Immunol. 147:86-95).

Alternatively, for example, the antibodies may be prepared by “repertoire cloning” (Persson et al. (1991) Proc. Nat. Acad. Sci. USA 88:2432-2436; and Huang and Stollar (1991) J. Immunol. Methods 141:227-236). Further, U.S. Pat. No. 5,798,230 describes preparation of human monoclonal antibodies from human B antibody-producing B cells that are immortalized by infection with an Epstein-Barr virus that expresses Epstein-Barr virus nuclear antigen 2 (EBNA2). EBNA2, required for immortalization, is then inactivated resulting in increased antibody titers.

Antibodies against FR-α may also be formed by in vitro immunization of peripheral blood mononuclear cells (“PBMCs”). This may be accomplished by any means known in the art, such as, for example, using methods described in the literature (Zafiropoulos et al. (1997) J. Immunological Methods 200:181-190).

Methods for producing antibody-producing cells may also include methods for developing hypermutable antibody-producing cells by taking advantage of the conserved mismatch repair (MMR) process of host cells. Dominant negative alleles of such genes, when introduced into cells or transgenic animals, increase the rate of spontaneous mutations by reducing the effectiveness of DNA repair and thereby render the cells or animals hypermutable. Blocking MMR in antibody-producing cells such as but not limited to: hybridomas; mammalian cells transfected with genes encoding for Ig light and heavy chains; mammalian cells transfected with genes encoding for single chain antibodies; eukaryotic cells transfected with Ig genes, can enhance the rate of mutation within these cells leading to clones that have enhanced antibody production, cells containing genetically altered antibodies with enhanced biochemical properties such as increased antigen binding, cells that produce antibodies comprising substantially only the antibody, and/or cells that are substantially free of FR-α binding competitors. The process of MMR, also called mismatch proofreading, is carried out by protein complexes in cells ranging from bacteria to mammalian cells. A MMR gene is a gene that encodes for one of the proteins of such a mismatch repair complex. Although not wanting to be bound by any particular theory of mechanism of action, a MMR complex is believed to detect distortions of the DNA helix resulting from non-complementary pairing of nucleotide bases. The non-complementary base on the newer DNA strand is excised, and the excised base is replaced with the appropriate base, which is complementary to the older DNA strand. In this way, cells eliminate many mutations that occur as a result of mistakes in DNA replication.

Dominant negative alleles may cause a MMR defective phenotype even in the presence of a wild-type allele in the same cell. An example of a dominant negative allele of a MMR gene is the human gene hPMS2-134, which carries a truncating mutation at codon 134. The mutation causes the product of this gene to abnormally terminate at the position of the 134th amino acid, resulting in a shortened polypeptide containing the N-terminal 133 amino acids. Such a mutation causes an increase in the rate of mutations, which accumulate in cells after DNA replication. Expression of a dominant negative allele of a mismatch repair gene results in impairment of mismatch repair activity, even in the presence of the wild-type allele. Any allele which produces such effect can be used. Dominant negative alleles of a MMR gene can be obtained from the cells of humans, animals, yeast, bacteria, or other organisms. Such alleles can be identified by screening cells for defective MMR activity. Cells from animals or humans with cancer can be screened for defective mismatch repair. Cells from colon cancer patients may be particularly useful. Genomic DNA, cDNA, or mRNA from any cell encoding a MMR protein can be analyzed for variations from the wild type sequence. Dominant negative alleles of a MMR gene can also be created artificially, for example, by producing variants of the hPMS2-134 allele or other MMR genes. Various techniques of site-directed mutagenesis can be used. The suitability of such alleles, whether natural or artificial, for use in generating hypermutable cells or animals can be evaluated by testing the mismatch repair activity caused by the allele in the presence of one or more wild-type alleles, to determine if it is a dominant negative allele. A cell into which a dominant negative allele of a mismatch repair gene has been introduced will become hypermutable. This means that the spontaneous mutation rate of such cells or animals is elevated compared to cells or animals without such alleles. The degree of elevation of the spontaneous mutation rate can be at least 2-fold, 5-fold, 10-fold, 20-fold, 50-fold, 100-fold, 200-fold, 500-fold, or 1000-fold that of the normal cell or animal. The use of chemical mutagens such as but limited to methane sulfonate, dimethyl sulfonate, 06-methyl benzadine, MNU, ENU, etc. can be used in MMR defective cells to increase the rates an additional 10 to 100 fold that of the MMR deficiency itself.

A polynucleotide encoding a dominant negative form of a MMR protein may be introduced into a cell. Preferably the cell produces anti-FR-α antibodies. The cells may produce an antibody comprising a heavy chain comprising an amino acid sequence of SEQ ID NO:4, 5, or 6 and a light chain comprising an amino acid sequence of SEQ ID NO:1, 2, or 3. The cells may also comprise a nucleic acid comprising a nucleotide sequence of SEQ ID NO:7 and/or a nucleotide sequence of SEQ ID NO:8. The dominant negative MMR gene can be any dominant negative allele encoding a protein which is part of a MMR complex, for example, PMS2, PMS1, MLH1, or MSH2. The dominant negative allele can be naturally occurring or made in the laboratory. The polynucleotide can be in the form of genomic DNA, cDNA, RNA, or a chemically synthesized polynucleotide.

The polynucleotide can be cloned into an expression vector containing a constitutively active promoter segment (such as but not limited to CMV, SV40, Elongation Factor or LTR sequences) or an inducible promoter sequence such as the steroid inducible pIND vector (Invitrogen), where the expression of the dominant negative MMR gene can be regulated. The polynucleotide can be introduced into the cell by transfection.

An immunoglobulin (Ig) gene, a set of Ig genes or a chimeric gene containing whole or parts of an Ig gene can be transfected into MMR-deficient cell hosts, the cell is grown and screened for clones with new phenotypes and/or genotypes. MMR-defective cells may be of human, primates, mammals, rodent, plant, yeast or of the prokaryotic kingdom. The gene encoding the Ig of the cell with the new phenotype or genotype may be isolated from the respective clone and introduced into genetically stable cells (i.e., cells with normal MMR) to provide clones that consistently produce the Ig. The method of isolating the Ig gene may be any method known in the art. Introduction of the isolated polynucleotide encoding the Ig may also be performed using any method known in the art, including, but not limited to transfection of an expression vector containing the polynucleotide encoding the Ig. As an alternative to transfecting an Ig gene, a set of Ig genes or a chimeric gene containing whole or parts of an Ig gene into an MMR-deficient host cell, such Ig genes may be transfected simultaneously with a gene encoding a dominant negative mismatch repair gene into a genetically stable cell to render the cell hypermutable.

Transfection is any process whereby a polynucleotide is introduced into a cell. The process of transfection can be carried out in a living animal, e.g., using a vector for gene therapy, or it can be carried out in vitro, e.g., using a suspension of one or more isolated cells in culture. The cell can be any type of eukaryotic cell, including, for example, cells isolated from humans or other primates, mammals or other vertebrates, invertebrates, and single celled organisms such as protozoa, yeast, or bacteria.

In general, transfection will be carried out using a suspension of cells, or a single cell, but other methods can also be applied as long as a sufficient fraction of the treated cells or tissue incorporates the polynucleotide so as to allow transfected cells to be grown and utilized. The protein product of the polynucleotide may be transiently or stably expressed in the cell. Techniques for transfection are well known. Available techniques for introducing polynucleotides include but are not limited to electroporation, transduction, cell fusion, the use of calcium chloride, and packaging of the polynucleotide together with lipid for fusion with the cells of interest. Once a cell has been transfected with the MMR gene, the cell can be grown and reproduced in culture. If the transfection is stable, such that the gene is expressed at a consistent level for many cell generations, then a cell line results.

Upon identification of the desired phenotype or trait the organism can then be genetically stabilized. Cells expressing the dominant negative alleles can be “cured” in that the dominant negative allele can be turned off, if inducible, eliminated from the cell, and the like such that the cells become genetically stable and no longer accumulate mutations at the abnormally high rate.

Cells that produce substantially only anti-FR-α antibodies or cells that are substantially free of FR-α binding competitors may be selected for cloning and expansion according to any known method for determining antibody specificity. An example of a method for determining antibody specificity is described in U.S. patent application Ser. No. 12/500,144 filed on Jul. 9, 2009, which is a divisional of U.S. patent application Ser. No. 11/056,776 filed on Feb. 11, 2005, which are incorporated in their entirety.

Nucleic acids encoding antibodies may be recombinantly expressed. The expression cells may include any insect expression cell line known, such as for example, Spodoptera frugiperda cells. The expression cell lines may also be yeast cell lines, such as, for example, Saccharomyces cerevisiae and Schizosaccharomyces pombe cells. The expression cells may also be mammalian cells such as, for example, hybridoma cells (e.g., NS0 cells), Chinese hamster ovary cells, baby hamster kidney cells, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TR1 cells, MRC 5 cells, and FS4 cells. Nucleic acids may be introduced into cell by transfection, for example. Recombinantly expressed antibodies may be recovered from the growth medium of the cells, for example.

The procedure for in vitro immunization may be supplemented with directed evolution of the hybridoma cells in which a dominant negative allele of a mismatch repair gene such as PMS1, PMS2, PMS2-134, PMSR2, PMSR3, MLH1, MLH2, MLH3, MLH4, MLH5, MLH6, PMSL9, MSH1, and MSH2 is introduced into the hybridoma cells after fusion of the splenocytes, or to the myeloma cells before fusion. Cells containing the dominant negative mutant will become hypermutable and accumulate mutations at a higher rate than untransfected control cells. A pool of the mutating cells may be screened, for example, for clones that are substantially free of FR-α binding competitors, clones that produce higher affinity antibodies, clones that produce higher titers of antibodies, or clones that simply grow faster or better under certain conditions. The technique for generating hypermutable cells using dominant negative alleles of mismatch repair genes is described, for example, in U.S. Pat. No. 6,808,894. Alternatively, mismatch repair may be inhibited using the chemical inhibitors of mismatch repair described by Nicolaides et al. in WO 02/054856 “Chemical Inhibitors of Mismatch Repair” published Jul. 18, 2002. The technique for enhancing antibodies using the dominant negative alleles of mismatch repair genes or chemical inhibitors of mismatch repair may be applied to mammalian expression cells expressing cloned immunoglobulin genes as well. Cells expressing the dominant negative alleles can be “cured” in that the dominant negative allele can be turned off if inducible, inactivated, eliminated from the cell, and the like, such that the cells become genetically stable once more and no longer accumulate mutations at the abnormally high rate.

### Pharmaceutical Compositions of Antibodies

A pharmaceutical composition of anti-FR-α antibodies may be used to treat a non-functioning pituitary adenoma according to the method of the disclosure. The pharmaceutical compositions may be used to inhibit or reduce growth of tumor cells in a patient. The compositions of antibodies may be substantially free of FR-α binding competitors. The pharmaceutical composition may be also formulated for administration by injection or infusion. An example of such a composition is farletuzumab.

Pharmaceutical compositions used in the method of the disclosure may further comprise another anti-tumor agent, such as a chemotherapeutic or cytotoxic agent. The antibody may be conjugated to the chemotherapeutic or cytotoxic agent. Suitable chemotherapeutic or cytotoxic agents include but are not limited to a radioisotope, including, but not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, and fissionable nuclides such as Boron-10 or an Actinide. The agent may also be a toxin or cytotoxic drug, including but not limited to ricin, modified Pseudomonas enterotoxin A, calicheamicin, adriamycin, 5-fluorouracil, and the like. Pharmaceutical compositions may also comprise an antifolate compound including but not limited to 5-fluoro-2′-deoxy-uridine-5′-monophosphate (FdUMP), 5-fluorouracil, leucovorin, ZD1649, MTA, GW1843U89, ZD9331, AG337, and PT523.

Pharmaceutical compositions used in the method may also be formulated with a pharmaceutically acceptable carrier or medium. Suitable pharmaceutically acceptable carriers include water, PBS, salt solution (such as Ringer's solution), alcohols, oils, gelatins, and carbohydrates, such as lactose, amylose, or starch, fatty acid esters, hydroxymethylcellulose, and polyvinyl pyrrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical carriers suitable for use can be those known in the art and are described, for example, in Pharmaceutical Sciences (17th Ed., Mack Pub. Co., Easton, Pa.).

### Methods for Treating Non-Functioning Pituitary Adenomas

The methods of the disclosure are suitable for use in humans and non-human animals identified as having a non-functioning pituitary adenoma associated with an increased expression of FR-α. Non-human animals that may benefit from the method of the disclosure include pets, exotic (e.g., zoo animals), and domestic livestock. In some embodiments, the non-human animals are mammals.

The method is suitable for use in a human or animal patient that is identified as having a non-functioning pituitary adenoma that is marked by increased expression of FR-α in the neoplasm in relation to normal tissues. Once such a patient is identified as in need of treatment for such a condition, the method may be applied to effect treatment of the condition. Tumors that may be treated include, but are not limited to non-functioning pituitary tumors. The tumors may include other benign tumors with an increased expression of FR-α.

Antibodies and derivatives thereof may be administered to subjects with non-functioning pituitary adenomas who have potentially resectable disease but whose tumor bulk does not warrant immediate or emergent surgery. The method is suitable for use before or after tumor resection. In some embodiments, the administration of antibodies and derivatives may be administered as a preoperative neoadjuvant intervention. In other embodiments, the antibodies may be administered after an incomplete resection of a tumor.

Antibodies and derivatives thereof may be administered orally in any acceptable dosage form such as capsules, tablets, aqueous suspensions, solutions or the like. The antibodies and derivatives thereof may also be administered parenterally including but not limited to: subcutaneous, intravenous, intramuscular, intra-articular, intra-synovial, intrasternal, intranasal, topically, intrathecal, intrahepatic, intralesional, and intracranial injection or infusion techniques. Generally, the antibodies will be intravenously or intraperitoneally, for example, by injection. The antibodies and derivatives may be administered alone or with a pharmaceutically acceptable carrier, including acceptable adjuvants, vehicles and excipients, for example, phosphate buffered saline.

The antibodies and derivatives may also be administered with one or more anti-tumor agents, for example, antifolate compounds. The antifolate compounds may include, but are not limited to 5-fluoro-2′-deoxy-uridine-5′-monophosphate (FdUMP); 5-fluorouracil (5-FU); L-5-formyltetrahydrofolate (“leucovorin”); N-[5-(N-(3,4-dihydro-2-methyl-4-oxoquinazolin-6-yl-methyl)-amino)-2-theny-1)]-L-glutamic acid (“ZD1649”; also known as “Tomudex”) (Jackman et al. (1991) Cancer Res. 51:5579-5586); N-(4-(2-(2-amino-4,7-dihydro-4-oxo-3H-pyrrolo[2,3-D]pyrimidin-5-yl)-ethyl-)-benzoyl]-L-glutamic acid (“multi-targeted antifolate” (MTA) also known as “LY231514,” “ALIMTA,” and “Pemetrexed”) (Taylor et al. (1992) J. Med. Chem. 35:4450-4454; Shih et al. (1997) Cancer Res. 57:1116-1123); (S)-2-(5)-(((1,2-dihydro-3-methyl-1-oxobenzo(f)quinazolin-9-yl)-methyl)-a-mino)-oxo-2-isoindolinyl)-glutaric acid (“GW1843U89”) (Hanlon and Ferone (1996) Cancer Res. 56:3301-3306); (2S)-2-{0-fluoro-p-[N-(2,7-dimethyl-4-oxo-3,4-dihydro-quinazolin-6-yl-met-hyl)-N-prop-2-ynyl)amino]benzamido}-4-(tetrazol-5-yl)-butyric acid (“ZD9331”) (Jackman et al. (1997) Clin. Cancer Res. 3:911-921); 3,4-dihydro-amino-6-methyl-4-oxo-5-(4-pyridylthio)-quinazoline (“AG337” also known as “Thymitaq”).

The anti-tumor agents may be administered before, after, or simultaneously with the anti-FR-α antibodies. The amounts of anti-tumor agents to be administered may be the dosages currently used, or may be increased or decreased, as can readily be determined by a physician based on achieving decreased tumor growth or tumor elimination without causing any untoward effects on the patient.

The effective dosage will depend on a variety of factors. It is well within the purview of a skilled physician to adjust the dosage for a given patient according to various parameters such as body weight, the goal of treatment, the highest tolerated dose, the specific formulation used, the route of administration and the like. Generally, dosage levels of between 10.0 mg/kg and 1.0 mg/kg per day of the antibody or derivative thereof are suitable. The initial dose may be larger than the subsequent doses. In some embodiments, the dose may be about 5.0 mg/kg initially and then 2.5 mg/kg per subsequent doses. Dosing may be as a bolus or an infusion. Dosages may be given once a day or multiple times in a day. Further, dosages may be given multiple times of a period of time. The doses may be given every 1-14 days.

Effective treatment may be assessed in a variety of ways. In one embodiment, effective treatment may be determined by a slowed or inhibited progression of tumor growth. In other embodiments, effective treatment is marked by shrinkage of the tumor (i.e., decrease in the size of the tumor determined, for example, using Response Evaluation Criteria in Solid Tumors (RECIST) available online through the National Cancer Institute Cancer Therapy Evaluation Program). In other embodiments, effective treatment may be marked by inhibition of metastasis of the tumor.

### EXAMPLES

### Example 1

The potential efficacy of an antibody, for example, farletuzumab, will be evaluated as a preoperative neoadjuvant intervention. Twelve subjects will be treated and if at least 3 subjects have a clinically significant response (defined as >15% decrease in tumor size (longest diameter), then additional subjects (e.g., 13 subjects) will be treated. All enrolled subjects will receive 3 cycles of intravenous farletuzumab (initial loading does 5.0 mg/kg followed by 2.5 mg/kg for all subsequent doses). Each cycle will be 4 weeks long. Farletuzumab will be administered as an intravenous infusion on days 1 and 15. This duration was chosen to allow sufficient interval of time to detect significant change in tumor bulk using anatomic imaging. Formal visual field exam by neuro-opthalmology will be done at baseline, at 6 weeks and 12 weeks after starting farletuzumab. Following completion of therapy, a repeat MRI scan will be performed to determine the degree of response.

Pituitary adenomas are slow growing, do not metastasize, and exert damage by local pressure on vital nerve structures. Based on previous experience with dopamine agonists and somatostatin analogs, a decrease in tumor size (longest diameter) by >15% after 3 months of therapy will be considered clinically significant. All subjects are expected to undergo appropriate surgical tumor resection within 2 weeks of the last dose of farletuzumab. However, subjects who achieve a partial response, defined as >30% decrease in tumor size (longest diameter) while on farletuzumab will be given the option to either continue therapy in the study discussed in example 2 for as long as the investigator feels that the subject is deriving benefit or proceed with surgery as planned. Prior to surgery, a hormonal pituitary panel will be performed.

### Example 2

To assess the efficacy of farletuzumab in controlling disease in subjects with an incompletely resected tumor, subjects with incompletely resected tumors or those with partial response to farletuzumab therapy (e.g., from those in example 1) will be allowed to resume intravenous farletuzumab intervention (initial loading dose 5.0 mg/kg followed by 2.5 mg/kg for all subsequent doses) therapy provided that:
1. it is not earlier than 8 weeks post surgery;
2. the subject showed evidence of stable disease or decrease in tumor size during the preoperative treatment period as assessed by restaging MRI scan;
3. the subject did not experience intolerable toxicity during prior farletuzumab therapy; and
4. the subject is fully recovered from surgery.
   For this purpose, subjects will be treated as per the preoperative or previous treatment schedule of farletuzumab once every 2 weeks in a 4-week cycle with restaging MRI scan after every 3 cycles and formal visual field testing every 6 cycles. Subjects receiving postoperative therapy may be allowed to continue farletuzumab for a total of 1 year if there is no intolerable toxicity or progressive disease. The study will continue for up to 1 year if there is no intolerable toxicity or progressive disease. The study will continue for up to 1 year from the date of enrollment of the last subject in example 2. In the case that subjects appear to be deriving benefits past one year, an extension protocol will be submitted for institutional approval.

### Example 3

The non-functioning pituitary adenomas treated by the method of the disclosure may be selected based on in vivo diagnosis of tumor receptor expression. This will allow normal tissues, lacking in folate receptors, to be spared toxicity associated with nontargeted drug therapy. Such selection may help to decrease toxicity and increase efficacy.

The in vivo assay of folate receptors in non-functioning pituitary adenomas can be evaluated using preoperative ^{99m}Tc-folate SPECT/CT and Western blot analysis (WBA) of surgical specimens as the standard. Fifty-six patients (29 men, 27 women; age range, 29-82 y) with clinically non-functioning pituitary adenomas on MRI underwent preoperative imaging using 666 MBq (18 mCi) of 99 mTc-folate. SPECT/CT images and whole-body and lateral head planar images were acquired approximately 2 h after injection. Surgical resection took place within a week. WBA on a portion of the excised specimens assessed folate receptor expression in 49 patients. It was found that planar imaging of ^{99m}Tc-folate had good sensitivity and specificity (81% and 72%, respectively), with a positive predictive value of 83%. The addition of SPECT/CT improved the sensitivity (94%), at some loss of specificity (61%), by qualitative assessment. SPECT/CT improved the clinician's confidence in the location of any uptake noted and, consequently, the confidence of diagnoses. Quantitative analysis of the SPECT/CT image showed similar sensitivity to planar imaging (81%) but higher specificity (83%). Galt et al. (2010) J Nucl Med 51:1716-1723.

### Combination of the Antibodies and Anti-Tumor Agents

For combination therapy, efficacy may be demonstrated in vitro using an essay and the antibodies. One of skill in the art may extrapolate dosages from the in vitro efficacy assays to determine a range of efficacy in patients. Furthermore, dosages of antibodies accepted in the art for administration can be matched with dosages accepted for various folate inhibitors and adjusted to achieve maximum benefit with the minimum dosage. One of skill in the art is able to adjust these dosages to achieve the desired effect with routine experimentation particularly with the guidance on dosage for antibodies provided above and the assay described for determining an effect in vitro.

What is claimed:

1. A method of treating a pituitary adenoma comprising administering farletuzumab to a subject diagnosed with a pituitary adenoma.

2. The method of claim 1, wherein said subject underwent a medical procedure resecting a portion of the pituitary adenoma.

3. The method of claim 2, where farletuzumab is administered before said medical procedure.

4. The method of claim 2, wherein farletuzumab is administered after said medical procedure.

5. The method of claim 2, wherein farletuzumab is administered before and after said medical procedure.

6. The method of claim 1, wherein farletuzumab is administered by intravenous infusion.

7. The method of claim 1, wherein farletuzumab is administered between 10.0 mg/kg and 1.0 mg/kg.

8. The method of claim 7, wherein farletuzumab is administered between 5.0 mg/kg and 2.5 mg/kg.

9. The method of claim 1, wherein farletuzumab is administered in combination with a different anti-tumor agent.

10. The method of claim 1, wherein the administering the farletuzumab induces a therapeutic effect.

11. The method of claim 10, wherein the therapeutic effect is an inhibition of growth of the adenoma.

12. The method of claim 10, wherein the therapeutic effect is a decrease in size of the adenoma.

13. A method of treating a pituitary adenoma comprising administering antibodies that specifically bind to alpha-folate receptor to a subject diagnosed with a pituitary adenoma.

14. The method of claim 13, wherein the antibodies are administered with a different anti-tumor agent.

15. The method of claim 13, wherein the administering the antibodies induces a therapeutic effect.

16. The method of claim 15, wherein the therapeutic effect is an inhibition of growth of the adenoma.

17. The method of claim 15, wherein the therapeutic effect is a decrease in size of the adenoma.


SEQUENCE LISTING (6 sequences)

SEQ ID NO: 1 - protein, 110 aa, Homo sapiens
DIQLTQSPSSLSASVGDRVTITCSVSSSISSNNLHWYQQKPGKAPKPWIYGTSNPASGVPSRFSGSGSGTDYTFTISSLQPEDIATYYCQQWSSYPYMYTFGQGTKVEIK

SEQ ID NO: 2 - protein, 217 aa, Homo sapiens
DIQLTQSPSSLSASVGDRVTITCSVSSSISSNNLHWYQQKPGKAPKPWIYGTSNPASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 236 aa, Homo sapiens
MGWSCIILFLVATATGVHSDIQLTQSPSSLSASVGDRVTITCSVSSSISSNNLHWYQQKPGKAPKPWIYGTSNPASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 119 aa, Homo sapiens
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 5 - protein, 449 aa, Homo sapiens
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 468 aa, Homo sapiens
MGWSCIILFLVATATGVHSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK